(19) United States
(12) Patent Application Publication
(10) Pub. No.: US 2026/0083915 A1
(43) Pub. Date: Mar. 26, 2026

(54) Autoinjector for Automatic Injection of a Product Into an Injection Site

(71) Applicant: Becton Dickinson France, Le Pont-de-Claix (FR)

(72) Inventors: Lionel Maritan, Bresson (FR); Franck Carrel, Saint Jean de Vaulx (FR); Michael Fiard, Eybens (FR)

(21) Appl. No.: 19/106,887

(22) PCT Filed: Aug. 11, 2023
(86) PCT No.: PCT/EP2023/072281

§ 371 Date: Feb. 26, 2025

(30) Foreign Application Priority Data
Sep. 5, 2022 (EP) 22306315.7

(51) Int. Cl. A61M 5/20; A61M 5/315; A61M 5/32

(52) U.S. Cl. CPC A61M 5/2033; A61M 5/31501; A61M 5/3245; A61M 2005/31508; A61M 2005/3247

(57) ABSTRACT

This autoinjector includes a housing extending along longitudinal axis A and configured to receive a medical container having a barrel defining a reservoir for containing a medical product, said barrel having a distal end provided with an injection needle and an opened proximal end configured to receive a plunger rod for pushing a stopper arranged inside the barrel. A needle cover is coupled to and axially movable with respect to said housing between a first extended position, a retracted position and a second extended position. A plunger rod is axially movable inside the housing between an initial position and an injection end position, the plunger rod being configured to push the stopper in order to expel the medical product when moving from the initial position to the injection end position. The autoinjector further includes biasing member for biasing the plunger rod in a distal direction towards the injection end position, and a retainer for maintaining the plunger rod in the initial position against the action of the biasing member, the retainer including a resilient blocking member radially deformable between a blocking position for blocking the plunger rod in the initial position, and a release position allowing for movement of the plunger rod in the distal direction. A lock ring is coupled to and axially movable with respect to the resilient blocking member, between a locking position for maintaining the resilient blocking member in the blocking position, and an unlocking position, axial movement of the lock ring from the locking to the unlocking position being caused by the needle cover moving from the first extended position to the retracted position.

### CROSS-REFERENCE TO RELATED APPLICATIONS

This application is the United States national phase of International Patent Application No. PCT/EP2023/072281 filed Aug. 11, 2023, and claims priority to European Patent Application No. 22306315.7 filed Sep. 5, 2022, the disclosures of which are hereby incorporated by reference in their entireties.

### BACKGROUND OF THE INVENTION

### Field of the Invention

The present invention relates to an autoinjector.

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction away from the user's hand, and the “proximal direction” is to be understood as meaning the direction toward the user's hand.

### Description of Related Art

Automatic injection devices are designed for automatic injection of a medical product into an injection site. Autoinjectors usually comprise a housing for receiving a medical container. The medical container has a barrel defining a reservoir for containing the medical product, the barrel having a distal end provided with an injection needle and an opened proximal end receiving a plunger rod for pushing a stopper. The opened proximal end is usually provided with a flange.

Autoinjectors also include a safety shield mechanism moving from an extended to a retracted position to respectively shield or unveil the needle and an injection mechanism for automatically injecting the medical into an injection site. The injection mechanism usually includes a plunger rod for pushing a stopper inside the barrel of the medical container, and an initially compressed spring for moving the plunger in the distal direction. Locking means are provided for maintaining the plunger rod in an initial position in which the plunger rod is axially blocked despite the action of the compressed spring. A release member is typically arranged to release the plunger from the locking means and allow the spring to push the plunger rod in the distal direction to perform injection. A predetermined displacement of the safety shield towards the retracted position is required to allow the release member to unlock the locking means and release the plunger rod.

The document EP2921191 discloses a device for automatic injection of a medical product into an injection site. The device includes a two-part assembly having an upper sub-assembly assembled to a lower sub-assembly. The lower sub-assembly receives a syringe provided with an injection needle. The injection needle is covered by a rigid needle shield. The device further includes a needle cover movable relative to the housing between an extended and a retracted position. The plunger rod is coupled to a compressed spring and maintained in an initial position by a deflectable retainer of an inner cylinder. A push button is arranged at a proximal end of the device for releasing the plunger rod by deflecting the retainer outwardly. Activation of the push button only becomes possible when the needle cover has moved a predetermined distance in the proximal direction towards the retracted position. Besides, activation of the push button requires the end user to press the push button. That is, the end user must perform a specific additional operation to activate the device. Moreover, this operation may not be so easy since the end user must in the same time exert a distal force on the push button and hold the autoinjector pressed against his/her skin.

The document US2021125536 discloses a power unit for use in an autoinjector. The document US2021121633 discloses a drug delivery device including a housing, a drug storage container, a plunger and a container holder configured to support the drug storage container. The document US2020164456 discloses an autoinjector including a case, a syringe carrier, a plunger and a drive spring.

There is therefore a need for an autoinjector allowing activation of the injection mechanism without requiring an additional operation from the end user.

Besides, in some prior art autoinjectors, the retainer that retains the plunger rod may creep over time under the stress of the compressed spring. Thus, there is also a need for an autoinjector that is less sensitive to material creep over long storage periods.

### SUMMARY OF THE INVENTION

An aspect of the invention is an autoinjector, for automatic injection of a product into an injection site, said autoinjector comprising:
a housing extending along a longitudinal axis A and configured to receive a medical container having a barrel defining a reservoir for containing a medical product, said barrel having a distal end provided with an injection needle and an opened proximal end configured to receive a plunger rod for pushing a stopper arranged inside the barrel,
a needle cover coupled to and axially movable with respect to said housing between a first extended position, in which the needle cover at least partially shields the injection needle, a retracted position, in which the needle cover moves proximally with respect to the housing, and a second extended position in which the needle cover moves back in the distal direction to shield the injection needle,
a plunger rod axially movable inside the housing between an initial position and an injection end position distally located relative to said initial position, the plunger rod being configured to push the stopper in order to expel the medical product when moving from the initial position to the injection end position,
biasing member for biasing the plunger rod in a distal direction towards the injection end position,
a retainer for maintaining the plunger rod in the initial position against the action of the biasing member, the retainer including a resilient blocking member radially deformable between a blocking position, in which the resilient blocking member axially abuts against a distal abutment surface of the plunger rod for blocking the plunger rod in the initial position, and a release position, in which the resilient blocking member is away from the distal abutment surface of the plunger rod to allow for movement of the plunger rod in the distal direction,
a lock ring coupled to and axially movable with respect to the resilient blocking member, between a locking position, in which the lock ring radially abuts against a lateral blocking surface of the resilient blocking member to maintain the resilient blocking member in the blocking position, and an unlocking position, in which the lock ring is axially shifted away from the lateral blocking surface of the resilient blocking member, axial movement of the lock ring from the locking to the unlocking position being caused by the needle cover moving from the first extended position to the retracted position.

The autoinjector of the invention allows easier activation of the injection mechanism. Indeed, there is no need to manually press a push button, since the push button has been replaced by a slidable lock ring which automatically moves to an unlocking position when the needle cover moves to the retracted position. The end user has no additional operation to perform, the lock ring slides automatically due to the needle cover being pressed against the user's skin.

The autoinjector of the invention may further include some or all of the features below.

In an embodiment, the needle cover has a pushing surface configured to abut against a distal abutment surface of the lock ring to move said lock ring in the unlocking position when the needle cover moves towards the retracted position. Thus, the needle cover directly abuts against the lock ring without any intermediate component. The activation of the autoinjector gets easier for the user.

In an embodiment, the pushing surface of the needle cover and the distal abutment surface of the lock ring are axially at a distance from each other before use of the autoinjector. That is, an axial gap exists between the pushing surface of the needle cover and the proximal abutment surface of the lock ring when the needle cover is in the first extended position and the lock ring is in the locking position.

In an embodiment, the blocking position of the resilient blocking member is a rest position. That is, the blocking member is not deformed in the blocking position, and deforms when moving to the release position. More specifically, the resilient blocking member extends parallel to the longitudinal axis A in the blocking position, and resilient blocking member radially deflects in the release position, so that an axial stop of the resilient blocking is shifted away from the distal abutment surface of the plunger rod which thus can move in the distal direction under the pressure of the biasing member.

In an embodiment, the retainer is an axial sleeve arranged within the housing and the resilient blocking member is an axially extending resilient blocking leg forming a distal end or a proximal end of said axial sleeve, said blocking leg including an inward radial protrusion, which defines an axial stop for abutting against the distal abutment surface of the plunger rod, and an opposite outward radial protrusion, which defines the lateral blocking surface for abutting against the lock ring.

Preferably, the retainer defines inner cavity for receiving the plunger rod. The inner cavity may also receive the biasing member. The biasing member may be arranged outside or inside the plunger rod.

Possibly, the retainer and the housing are made of a single piece.

Preferably, the lock ring extends around the retainer. The lateral blocking surface of the resilient blocking member is an outer lateral blocking surface and the lock ring has a corresponding inner lateral blocking surface for abutting against said outer lateral blocking surface in the locking position.

In an embodiment, the lock ring includes a proximal portion and a distal portion separated by a distal shoulder, and the distal portion has a larger inner diameter D1 than the inner diameter D2 of the proximal portion. Thus, the proximal portion prevents outward deformation of the retainer while the distal portion allows for outward deformation of the retainer. The proximal portion may be configured to receive the resilient blocking member when the lock ring is in the locking position, while the distal portion may be configured to receive the resilient blocking member when the lock ring is in the unlocking position.

In an embodiment, the distal shoulder of the lock ring and the outward radial protrusion of the resilient blocking member of the retainer are at an axial distance from each other when the lock ring is in the unlocking position and the resilient blocking member is in the release position. Thus, the distal shoulder does not prevent the resilient blocking member to deflect towards the release position.

In an embodiment, the outward radial protrusion of the resilient blocking member includes a proximal stop configured to abut against the distal shoulder of the lock ring for preventing the lock ring to move back to the blocking position.

In an embodiment, the lock ring includes one or more axial protection walls defining a radial room for receiving the resilient blocking member in the release position. The protection walls may form the distal portion of the lock ring. The protection walls avoid interference of the resilient blocking member with other parts of the autoinjector so that the injection process can be successfully completed. The protection walls may extend only partially in the circumferential direction, i.e. they do not extend all around the longitudinal axis A. Preferably, the distal abutment surface of the lock ring may be arranged at a distal end of the distal portion, i.e. the protection walls. Besides, the distal portion (the protection walls) may be dimensioned such that a distal end of the lock ring is distally located with respect to a distal end of the resilient blocking member when the resilient blocking member is in the release position and the lock ring is in the unlocking position. Thus, the resilient blocking member does not interfere with the needle cover.

In an embodiment, the lock ring defines an inner cavity for receiving the retainer, said inner cavity defining a centering surface for sliding against the retainer and guiding the movement of the lock ring along the longitudinal axis A, and an axial groove defining a lateral blocking surface for blocking the resilient blocking member of the retainer in the blocking position. The inner cavity of the lock ring has a proximal opening allowing the lock ring to be mounted around the retainer and an opposite distal opening allowing the plunger rod to extend therethrough and move towards the injection end position. The inner cavity may be arranged at the proximal portion of the lock ring.

In an embodiment, the autoinjector includes maintaining means for maintaining the lock ring assembled to the retainer before activation of the autoinjector.

In an embodiment, the maintaining means include at least a first blocking feature for blocking axial movement of the lock ring in the distal direction, and a second blocking feature for blocking axial movement of the lock ring in the proximal direction when a proximal force exerted on said lock ring is below a predetermined threshold, i.e. before activation of the autoinjector. The second abutment may be formed by a second dot of the lock ring axially abutting against a second stop of the retainer when the lock ring is in the locking position.

In a possible embodiment, the inner cavity further includes an axially extending flexible leg which has a blocking feature configured for engaging a corresponding blocking feature of the retainer to maintain the lock ring in the locking position when a proximal pushing force exerted by the needle cover on the lock ring is below a predetermined threshold. Thus, the autoinjector is not activated when the autoinjector falls down cap upward on the floor. The connecting feature thus allows for passing drop tests.

In an embodiment, the distal abutment surface of the lock ring is arranged at a distal end of said lock ring and the pushing surface of the needle cover is arranged at a proximal end of said needle cover. More specifically, the distal abutment surface of the lock ring may be the distal-most surface of the lock ring. Likewise, the pushing surface of the needle cover may be the proximal-most surface of the needle cover.

In an embodiment, the needle cover has a radial tab engaged in a first slot of a rotatable cam, and the radial tab has a proximal surface which at least partially forms the pushing surface of the needle cover.

In an embodiment, the lock ring includes has a guiding member for preventing rotation of said lock ring around the longitudinal axis A. The guiding member may include a groove, which may be arranged at an outer lateral surface the lock ring, for receiving an axial guiding rib of the housing. The guiding member of the lock ring may be arranged at the proximal portion of said lock ring.

In an embodiment, the housing includes a connector for fixedly connecting the retainer to the housing, said connector delimiting a cutout for receiving the lock ring and a guideway for engaging the guiding member of the lock ring.

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIG. 1 is an exploded view of an autoinjector according to an embodiment of the invention,

FIGS. 2A and 2B are perspective cross-section views of a top body of an autoinjector according to an embodiment of the invention,

FIG. 3 is a perspective view of a needle cover of an autoinjector according to an embodiment of the invention,

FIG. 4A is a perspective view of a lock ring of an autoinjector according to an embodiment of the invention,

FIG. 4B is a perspective cross-section view of a lock ring of an autoinjector according to an embodiment of the invention,

FIG. 4C is a top view of a lock ring of an autoinjector according to an embodiment of the invention,

FIGS. 5A and 5B are cross-section views of an autoinjector according to an embodiment of the invention, in an blocking position of the lock ring,

FIGS. 6A and 6B are cross-section views of an autoinjector according to an embodiment of the invention, in a release position of the lock ring,

FIGS. 7A and 7B are, respectively, a cross-section and a perspective view of a plunger rod of an autoinjector according to an embodiment of the invention,

FIGS. 8A and 8B are perspective views of, respectively, a lock ring and a retainer of an autoinjector according to an embodiment of the invention,

FIG. 9A is a cross-section view of an autoinjector according to an embodiment of the invention,

FIG. 9B is a perspective view of a lock ring of an autoinjector according to an embodiment of the invention,

FIG. 9C is a partial perspective view of a retainer of an autoinjector according to an embodiment of the invention,

FIG. 10A is a cross-section view of an autoinjector according to an embodiment of the invention,

FIG. 10B is a perspective view of the lock ring and the retainer of an autoinjector according to an embodiment of the invention,

FIG. 11A is a cross-section view of an autoinjector according to an embodiment of the invention, the lock ring being in the locking position,

FIG. 11B is a detail of the autoinjector of FIG. 11A,

FIG. 11C is a cross-section view of an autoinjector according to an embodiment of the invention, after a 90° rotation from FIG. 11A,

FIG. 11D is a detail of FIG. 11C,

FIG. 11E is a cross-section of an autoinjector according to an embodiment of the invention, the lock ring being in the unlocking position,

FIG. 11F is a detail of FIG. 11E

FIG. 11G is a cross-section view of an autoinjector according to an embodiment of the invention, after a 90° rotation from FIG. 11F,

FIG. 11H is a perspective and partial cross-section view of an autoinjector according to an embodiment of the invention,

FIG. 11I is a perspective view of a lock ring of an autoinjector according to an embodiment of the invention.

### DESCRIPTION OF THE INVENTION

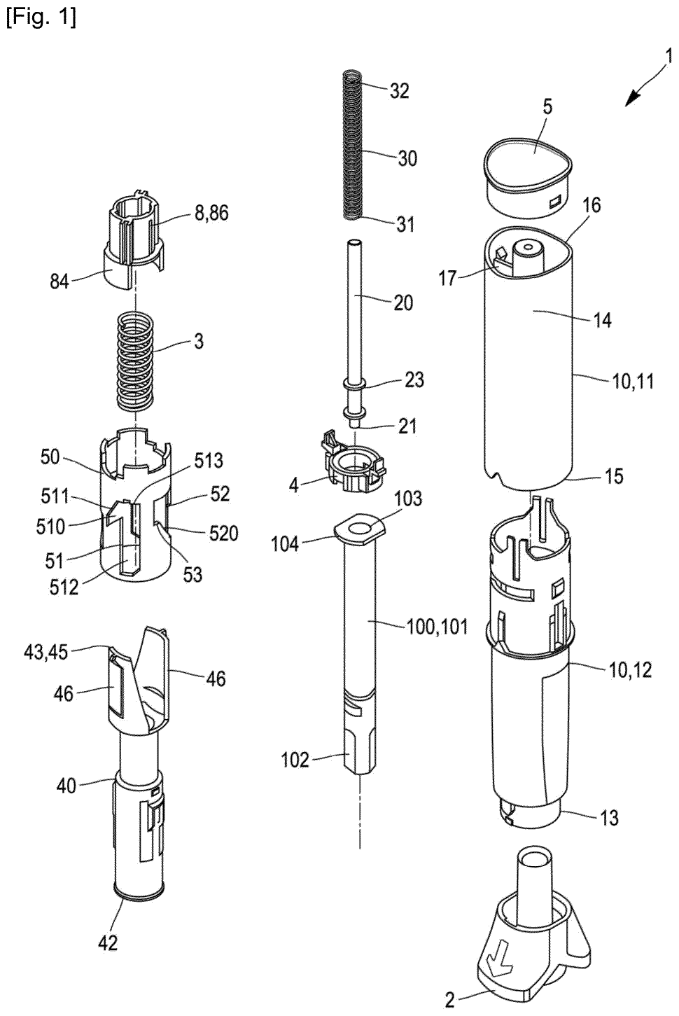

With reference to FIG. 1 is shown an autoinjector 1 according to an embodiment of the invention. The autoinjector 1 is designed for automatic injection of a product into an injection site. The autoinjector 1 extends along a longitudinal axis A. The autoinjector 1 includes a housing 10 comprising a top body 11 and a bottom body 12 assembled to each other by any appropriate securing means such as, for instance, snap-fitting means.

The bottom body 12 is configured to receive a medical container 100 such as a syringe, for instance a prefilled syringe. The medical container 100 has a tubular barrel 101 defining a reservoir for containing a medical product to be injected. The barrel 101 has a distal end provided with an injection needle (not shown) and a neddle shield 102 removably attached to said distal end for protecting and sealing the injection needle. The barrel 101 has an opened proximal end 103 which may be provided with a flange 104 and which is configured to receive a plunger rod 20 urged in the distal direction by an injection spring 30. The plunger rod 20 is configured to push a stopper 105 arranged inside the barrel 101, thereby expelling the medical product.

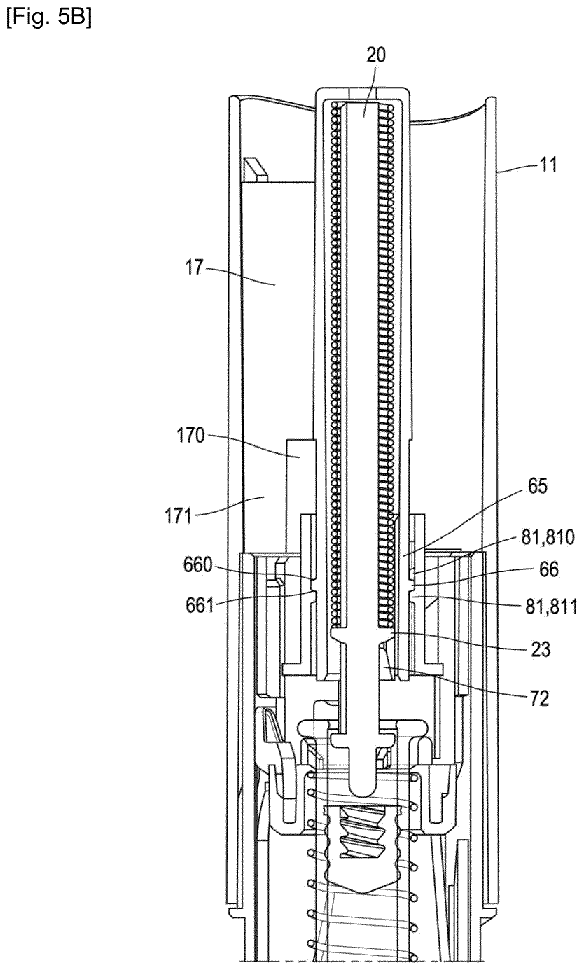

To that end, the plunger rod 20 is axially movable inside the housing 10 between an initial position (FIG. 5A or 5B), and an injection end position distally located with regard to the initial position. In the initial position, the injection spring 30 is compressed; a distal end 21 of the plunger rod 20 may be axially away from the stopper 105. In the injection end position, the injection spring 30 is extended; the distal end 21 of the plunger rod 20 may press the stopper 105 against a distal end 106 of the reservoir defined by the barrel 101. Movement of the plunger rod 20 from the initial position to the injection end position is caused by the injection spring 30 pushing the plunger rod 20 in the distal direction.

The plunger rod 20 includes a distal abutment surface 22 which may be arranged at a distal side of an outer flange 23 for abutting against an axial stop 71, thereby axially blocking the plunger rod 20 in the initial position. A proximal side of the outer flange 23 may serve as a support for a distal end 31 of the injection spring 30. In the embodiment illustrated in FIGS. 1-6B, the injection spring 30 extends outside and around the plunger rod 20.

The autoinjector 1 also comprises a cap 2 removably attached to a distal end 13 of the bottom body 12, a needle cover 40 arranged within the bottom body 12, a safety spring 3 for urging the needle cover 40 in a distal direction, and a cam 50 pivotally mounted around the needle cover 40, said cam 50 having a first slot 51 engaged by a radial tab 41 of the needle cover 40 and having a second slot 52 engaged by a ring 4 secured to the medical container 100 for blocking axial movement of the medical container 100 in the distal direction before activation of the autoinjector 1.

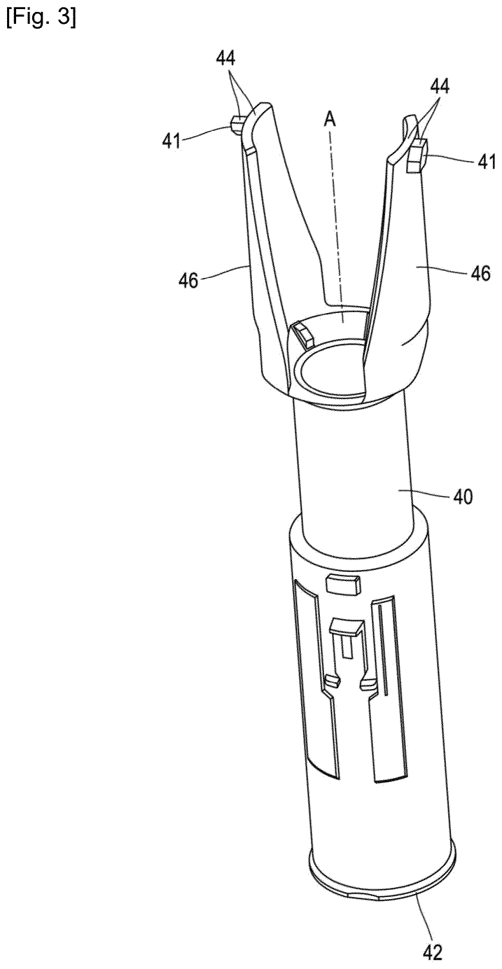

FIG. 3 illustrates the needle cover 40. The needle cover 40 is axially movable along the longitudinal axis A with respect to the bottom body 12 between a first extended position (pre-use position), in which the needle cover 40 at least partially or completely shields the injection needle, a retracted position (injection position), proximally located relative to said first extended position, in which the needle cover 40 moves inside the autoinjector 1 to trigger the injection, and a second extended position (safety position) in which the needle cover 40 moves back in the distal direction to safely shield the injection needle. The needle cover 40 has a distal end 42 designed to be pressed against an injection site, an opposite proximal end 43 defining a pushing surface 44 whose function will be explained below in further detail, and one or more radial tabs 41 for engaging the first slot 51 of the rotatable cam 50. Movement of the needle cover 40 from the first extended position to the retracted position is caused by the user pressing the distal end 42 of the needle cover 40 against the injection site, and movement of the needle cover 40 from the retracted position to the safety position is due to the safety spring 3 pushing the needle cover 40 back in the distal direction.

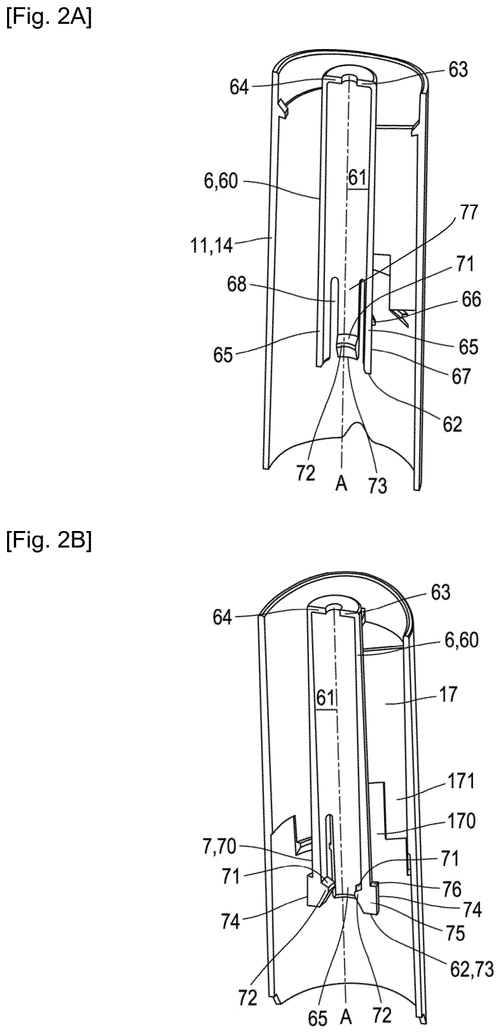

FIGS. 2A and 2B illustrate the top body 11. The top body 11 has a lateral wall 14, an opened distal end 15, and a closed proximal end 16. In the illustrated embodiment, the proximal end 16 of the top body 11 is closed by a top cap 5 fixed to the top body 11. The top cap 5 may either be a separate component that may be secured to the top body 11 by appropriate securing means, such as snap-fitting means, or may be made of a single piece with the top body 11. Still with reference to FIGS. 2A and 2B, the top body 11 includes a retainer 6 configured for retaining the plunger rod 20 in the initial position before activation of the autoinjector 1. The retainer 6 may be in the form of a tubular sleeve 60 secured to the lateral wall 14 by connectors 17. The sleeve 60 and the top body 11 may be made of a single piece. Thus, the retainer 6 is fixed relative to the housing 10.

The tubular sleeve 60 defines an inner cavity 61 for receiving the plunger rod 20 and the injection spring 30. The sleeve 60 includes an opened distal end 62 providing a passage for the plunger rod 20, and an opposite proximal end 63 which may be closed by a transversal wall 64 for providing support to a proximal end 32 of the injection spring 30.

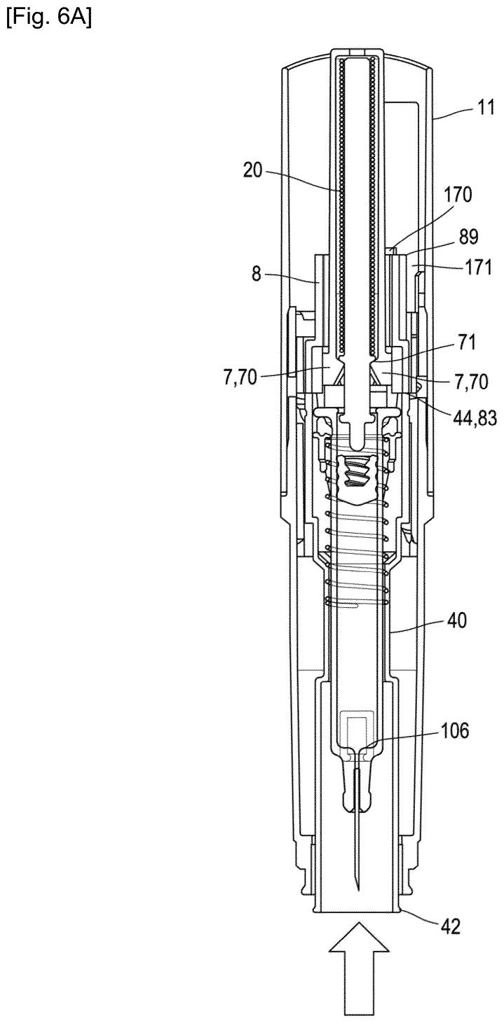

The distal end 62 of the sleeve 60 has one or more resilient blocking members 7 which may be in the form of axially extending resilient blocking legs 70 for blocking the plunger rod 20 in the initial position. To that end, the blocking legs 70 include an axial stop 71 for abutting against the distal abutment surface 22 of the plunger rod 20 in the initial position (FIG. 5A). The blocking legs 70 are movable between a blocking position which is a rest position (FIG. 5A), in which their axial stop 71 axially abuts against the distal abutment surface 22 of the plunger rod 20, and a release position which is a deformed position (FIG. 6B), in which the blocking legs 70 radially outwardly deflect so that their axial stop 71 is shifted away from the path of the plunger rod 20. Movement of the blocking legs 70 from the rest position to the deformed position is due to the injection spring 30 pushing the plunger rod 20 in the distal direction.

The axial stop 71 of the blocking legs 70 may be arranged at a proximal side of an inward radial protrusion 72, which is preferably located at a distal end 73 of the blocking legs 70. The proximal end 77 of the blocking legs 70 may be fixed to the sleeve 60. The axial stop 71 may be angled with regard to the longitudinal axis A to ease passage of the plunger rod 20 over the axial stop 71 when the autoinjector 1 is activated. The distal abutment surface 22 of the plunger rod 20 may be angled too, and may even be parallel to the axial stop 71 so that they are complementarily shaped. In the illustrated embodiment, the axial stop 71 and the distal abutment surface 22 have frustoconical shapes.

The blocking legs 70 may include a lateral blocking surface 74, which may be defined by an outward radial protrusion 75, for abutting against a lock ring 8, thereby maintaining the blocking legs 70 in their rest position. The lateral blocking surface 74 may be parallel to the longitudinal axis A so that the lock ring 8 can slide in the proximal direction. The outward radial protrusion 75 may be arranged radially opposite the inward radial protrusion 72, i.e. at the same level as the inward radial protrusion 72. The outward radial protrusion 75 is configured to engage axial grooves 80 of the lock ring 8 as will be seen below.

The tubular sleeve 60 may include several blocking legs 70 regularly distributed in a circumferential direction, for example two diametrically opposite blocking legs 70 although this number may vary. The tubular sleeve 60 may also include one or more axially extending centering legs 65, such as two diametrically opposite centering legs 65, for providing support for the lock ring 8 so that the lock ring 8 can be centered with respect to the central longitudinal axis A and can slide along the sleeve 60. The centering legs 65 are devoid of any inward protrusion and thus do not hamper axial movement of the plunger rod 20. The centering legs 65 may include a connecting feature 66, such a dot protruding from an outer lateral surface 67 of the centering legs 65, for engaging a corresponding connecting feature 81 of the lock ring 8, as will be explained below in further detail. The dot may have a rounded shape. The centering legs 65 and the blocking legs 70 may be separated from each other by axial slits 68, as visible in FIG. 2A or 2B.

Still with reference to FIGS. 2A and 2B, the connectors 17 may be in the form of radial walls extending parallel to the longitudinal axis A. They may define an axial cutout 170 for allowing the lock ring 8 to slide therein, and a guideway 171 in the form of an axial rib, which may extend along the inner surface of the lateral wall 14 of the top body 11, for guiding the proximal movement of the lock ring 8.

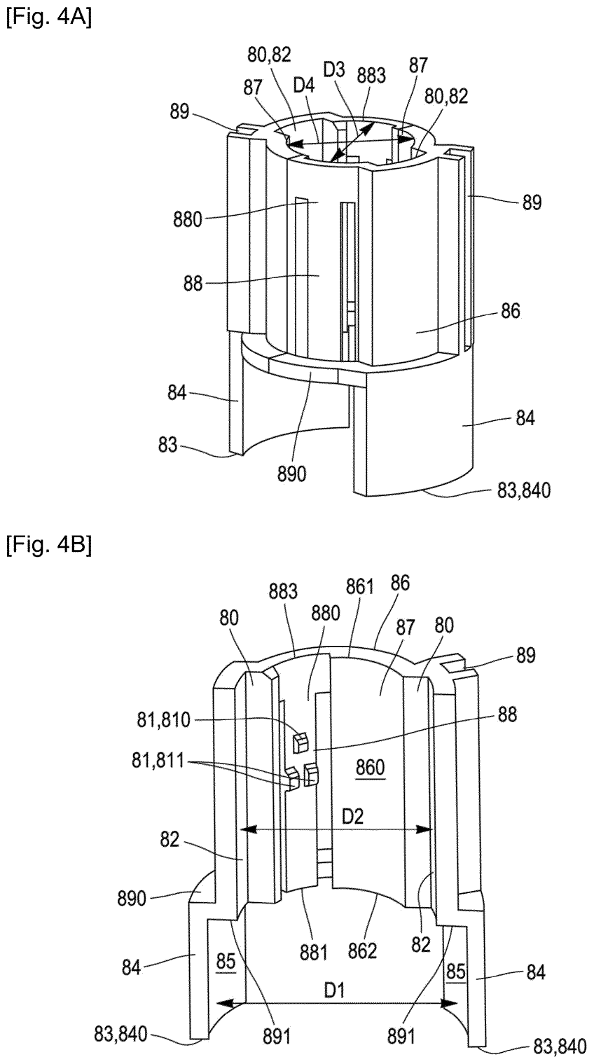

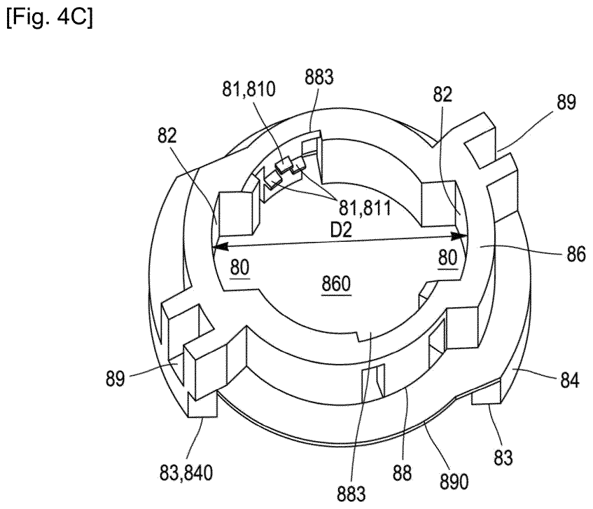

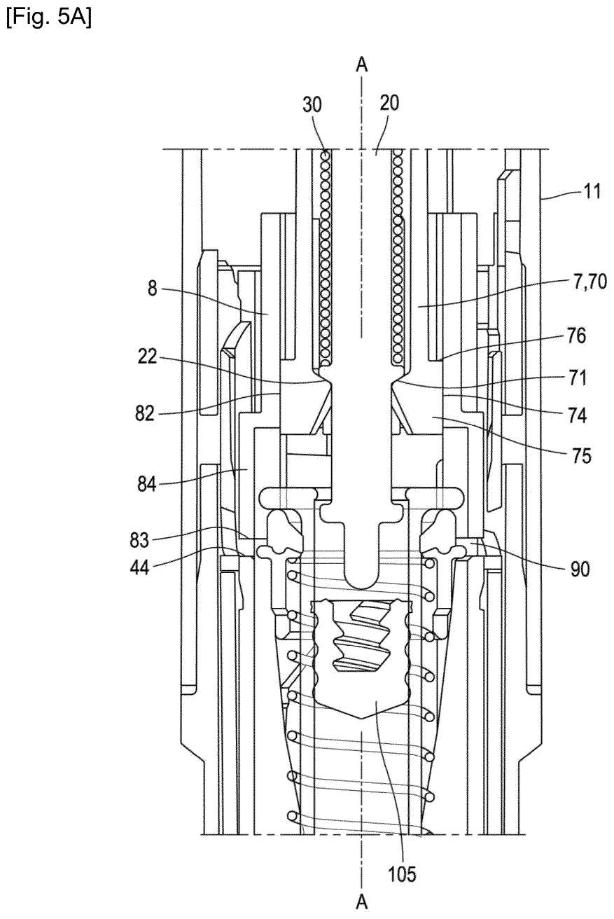

FIGS. 4A-4C illustrate the lock ring 8. The lock ring 8 is arranged on the retainer 6 for keeping the axial stop 71 of the retainer 6 in abutment against the distal abutment surface 22 of the plunger rod 20. The lock ring 8 thus helps maintain the plunger rod 20 in the initial position despite the action of the injection spring 30. The needle cover 40 and the lock ring 8, may be separate components. Likewise, the retainer 6 and the lock ring 8 are separate components.

The lock ring 8 is coupled to and axially movable with respect to the sleeve 60 between a locking position (FIG. 5A), in which a lateral blocking surface 82 of the lock ring 8 abuts against the lateral blocking surface 74 of the retainer 6 to prevent the blocking legs 70 from moving to their release position, and an unlocking position, proximally located with respect to the locking position, in which the lock ring 8 allows the blocking legs 70 for moving to their release position (FIGS. 6A, 6B). The autoinjector 1 is devoid of any spring that would bias the lock ring 8 in the distal or proximal direction. That is, apart from the maintaining means that temporarily maintain the lock ring 8 in the locking position, and the needle cover 40 that is configured to push the lock ring 8 in the proximal direction, the lock ring 8 is free to slide along the sleeve 60.

Movement of the lock ring 8 from the locking position to the unlocking position is caused by the pushing surface 44 of the needle cover 40 abutting against a distal abutment surface 83 of the lock ring 8. This occurs when the needle cover 40 moves towards the retracted position. In order to increase the reliability of the contact between the needle cover 40 and the lock ring 8, the pushing surface 44 of the needle cover 40 may be at least partially defined by the top (proximal) surface of the radial tabs 41, as visible in FIG. 3. That is, the top surface of the radial tabs 41 is at the same level as the pushing surface 44. Preferably, the pushing surface 44 is the proximal-most surface of the needle cover 40 and may be arranged at the proximal end 45 of two proximally extending legs 46 of said needle cover 40. The needle cover 40 directly pushes the lock ring 8, i.e. is in direct contact against the lock ring 8 without any intermediate component that would transmit the axial movement of the needle cover 40 to the lock ring 8.

With reference to FIGS. 4A-4C, the distal abutment surface 83 of the lock ring 8 may be arranged at a distal end 840 of one or more, for instance two diametrically opposite, axially extending protection walls 84. The protection walls 84 are designed to delimit a radial room 85 for receiving the deformed blocking legs 70 so that they do not hamper movement of other components such as the needle cover 40. The distal abutment surface 83 may be the distal-most surface of the lock ring 8. The distal abutment surface 83 of the lock ring 8 may be flat, orthogonal to the longitudinal axis A, and may longitudinally extend in a circumferential direction.

As illustrated in FIG. 5A, the needle cover 40 in the first extended position and the lock ring 8 in the locking position may be axially separated by a predetermined axial gap 90. The axial gap 90 allows for assembling of the autoinjector 1 without having the lock ring 8 being pushed by the needle cover 40 during this assembly. It should be noted that the needle cover 40 directly comes in abutment against the lock ring 8, i.e. without any intermediate part that would be arranged between the needle cover 40 and the lock ring 8. This direct contact permits to reduce the force required to trigger the injection and further prevents any axial looseness when activating the autoinjector 1. The pushing surface 44 of the needle cover 40 may extend in a circumferential direction, apart from the top surface of the tabs 41 which may define a radial portion of the pushing surface 44 for ensuring abutment between the needle cover 40 and the lock ring 8.

Still with reference to FIGS. 4A-4C, the lock ring 8 includes a tubular wall defining an inner cavity 86 for receiving the retainer 6. The inner cavity 86 leads to a proximal opening 861 allowing insertion of the retainer 6 and to an opposite distal opening 862 providing a passage for the plunger rod 20 moving towards the injection end position. An inner side of the tubular wall may define one or more centering surfaces 87, such as two diametrically opposite centering surfaces 87, which are complementarily shaped with the centering legs 65 of the retainer 6 for properly positioning the lock ring 8 with respect to the longitudinal axis A during assembly of the autoinjector 1. The tubular wall further defines one or more, for instance two diametrically opposite, axial grooves 80 whose bottom delimits the lateral blocking surfaces 82 of the lock ring 8. The axial grooves 80 are thus shaped to receive the outward radial protrusion 75 of the blocking legs 70 and allow translation of the lock ring 8 along the blocking legs 70. The diameter D2 defined between the lateral blocking surfaces 82 is lower than the diameter D1 between the two protection walls 84. Also, the axial length of the distal portion (the protection walls 84) of the lock ring 8 may be shorter than the axial length of the proximal portion (tubular wall 86) of the lock ring 8.

The autoinjector 1 may include maintaining means for temporarily maintaining the lock ring 8 in the locking position before use of the autoinjector 1. The maintaining means may be configured to release the lock ring 8 so that the lock ring 8 moves from the locking position to the unlocking position, as soon as a predetermined proximal force is exerted (by the needle cover 40) on the lock ring 8.

In the embodiment illustrated in FIGS. 4A-4C and 5B, the maintaining means include a first blocking feature 660, 810 for blocking axial movement of the lock ring 8 in the distal direction, and a second blocking feature 661, 811 for blocking axial movement of the lock ring 8 in the proximal direction when a proximal force exerted on said lock ring 8 is below a predetermined threshold.

More specifically, the first blocking feature includes one proximally located protrusion or first dot 810, and a proximal side or first stop 660 of a protrusion 66 of the retainer 6. The second blocking feature includes two distally located protrusions or second dots 811, and a distal side or second stop 661 of the protrusion 66 of the retainer 6. The first dot 810 and the second dots 811 may circumferentially be shifted from each other; they do not axially face each other and may thus form the vertices of a triangle. These blocking features 66, 81 form snap-fitting or clipping means for removably securing the lock ring 8 to the retainer 6. That is, the blocking features 66, 81 temporarily maintain the lock ring 8, in the locking position, assembled to the retainer 6 before activation of the autoinjector 1.

More specifically, the first dot 810 prevents disassembly of the lock ring 8 and the retainer 6, while the second dots 811 prevents the lock ring 8 to move to the unlocking position, for instance when the autoinjector 1 falls down on the floor (for example during a drop test “cap upward”). The second dots 811 thus prevent inadvertent activation of the autoinjector 1. The blocking features 81 of the lock ring 8 and the blocking feature 66 of the retainer 6 are however configured to disengage when the needle cover 40 pushes against the lock ring 8 during use of the autoinjector 1. To that end, the dots 810, 811 may have a rounded shape. Besides, the second dots 811 may be arranged to abut against the blocking feature 66 of the retainer 6 when the lock ring 8 is in the locking position. As a result, the user feels a resistance when the needle cover 40 comes in abutment against the lock ring 8, i.e. as soon as the needle cover 40 begins to push the lock ring 8 away from the locking position. This resistance informs the user that a further push of the autoinjector 1 against the injection site will trigger the device and the injection process. This also helps prevent activation of the autoinjector 1 during a drop test.

Also, the lock ring 8 may include an axially extending flexible leg 88 and the blocking features 81 of the lock ring 8 may be arranged on said axially extending flexible leg 88. The flexible leg 88 is configured to deflect radially outwardly so that the second dots 811 may pass over the blocking feature 66 of the retainer 6. The flexible leg 88 may have a fixed proximal end 880 and an opposite free distal end 881. As can be seen in FIG. 4C, the flexible leg 88 may be arranged in an axial recess 883. The lock ring 8 may include one or more, for instance two diametrically opposite, flexible legs 88. The diameter D3 between the flexible legs 88 may be greater than the diameter D4 between the centering surfaces 87 and lower than the diameter D2 between the lateral blocking surfaces 82 of the lock ring 8.

The lock ring 8 cannot rotate around the longitudinal axis A and moves in the axial direction only. The lock ring 8 further includes a guiding member 89 in the form of an axially extending guiding groove defined by two axial ribs 890 protruding from an outer side of the lateral wall 86 of the lock ring 8 for guiding the sliding movement of the lock ring 8 with respect to the sleeve 60 and the top body 11. The guiding member 89 also prevents rotation of the lock ring 8 around the longitudinal axis A. The groove 89 is configured to engage the axial rib 171 of the connectors 17 of the top body 11. The lock ring 8 includes one or more guiding members 89, for example two diametrically opposite guiding members 89 as illustrated in FIG. 4C. The grooves may have an opened proximal end and a distal end closed by a radial flange 890 of the lock ring 8.

The lateral wall 86 is arranged proximal to the protection walls 84 of the lock ring 8 such that the lateral wall 86 may form a proximal portion of the lock ring 8 and the protection walls 84 may form a distal portion of said lock ring 8. The lateral wall 86 and the protection walls 84 may be separated by the radial flange 890 which defines a distal shoulder 891. In the unlocking position, the distal shoulder 891 of the lock ring 8 may be axially away from the outward radial protrusion 75 of the retainer 6, as illustrated in FIG. 6B, so that the distal shoulder 891 does not hinder movement of the blocking legs 70 towards their release (deformed) position. More specifically, the distal shoulder 891 is axially away from a proximal stop 76 defined at a proximal side of the outward radial protrusion 75 of the blocking legs 70. This proximal stop 76 is configured to abut against the distal shoulder 891 of the lock ring 8 for preventing the lock ring 8 to move back to the blocking position when the needle cover 40 moves away from the lock ring 8 towards the safety position.

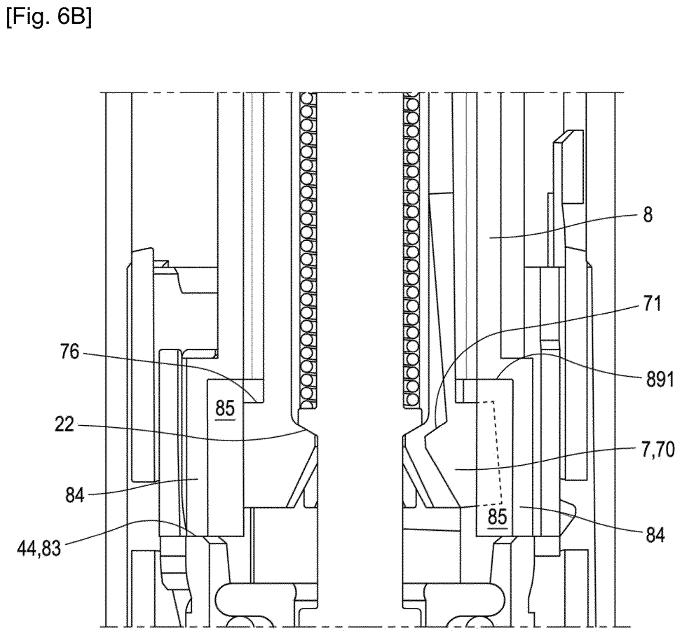

FIGS. 7A and 7B illustrate a plunger rod 20 according to another embodiment of the autoinjector 1. Similar features with the other embodiments are designated by the same numeral references. The plunger rod 20 of FIGS. 7A and 7B is similar to the plunger rod 20 of FIGS. 1-6B, except that the injection spring 30 is arranged within an inner cavity 24 of the plunger rod 20. A distal end 31 of the injection spring 30 may abut against the distal end 240 of the inner cavity 24 of the plunger rod 20. The distal abutment surface 22 of the plunger rod 20 is here arranged at a distal side of a radial window 25. The plunger rod 20 may include two diametrically opposite radial windows 25. The inward radial protrusion 72 of the blocking legs 70 is shaped to engage the radial windows 25 of the plunger rod 20.

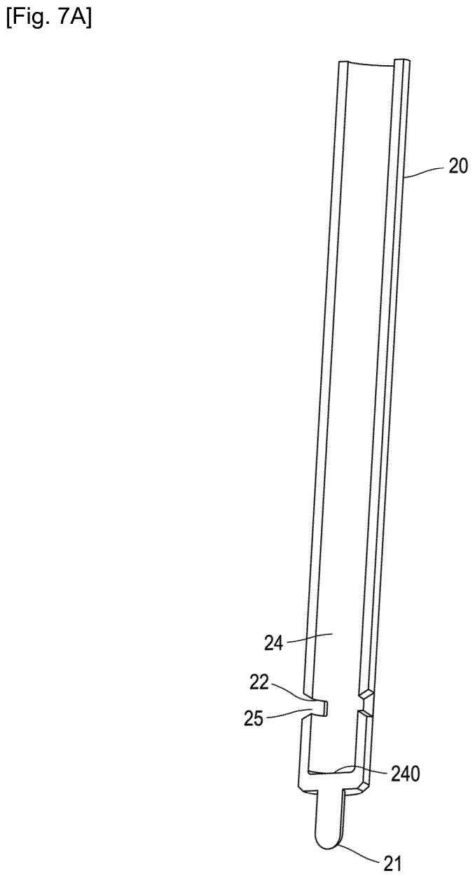

FIGS. 8A and 8B illustrate the maintaining means of a lock ring 8 and a retainer 6 according to another embodiment. The maintaining means include a first blocking feature 660, 810 for blocking axial movement of the lock ring 8 in the distal direction, and a second blocking feature 661, 811 for blocking axial movement of the lock ring 8 in the proximal direction when a proximal force exerted on said lock ring 8 is below a predetermined threshold. In this embodiment, the maintaining means and the lock ring 8 (preferably each flexible leg 88) includes only one second stop 811, instead of two. The first dot 810 and the second dot 811 may be circumferentially shifted with one another (i.e. not facing each other in the axial direction). It is contemplated that the flexible legs 88 may have two opposite fixed ends 880, 881, instead of one fixed proximal end 880 and one free distal end 881. Besides, the retainer 6 includes two protrusions 66, instead of one. The two protrusions 66 of the retainer 6 form a first dot 660 and a second dot 661, which may protrude from an outer surface of the centering legs 65. Preferably, as visible in FIG. 8B, the first dot 660 is arranged on one of the two centering legs 65, and the second dot 661 is arranged on the other one of the two diametrically opposite centering legs 65. The first dot 660 of the retainer 6 is configured to proximally abut against the first dot 810 of the lock ring 8 for preventing distal movement of the lock ring 8 (and thus disassembly of the lock ring 8 form the retainer 6). The second dot 661 of the retainer 6 is configured to distally abut against the second dot 811 of the lock ring 8 for preventing inadvertent proximal movement of the lock ring 8 (and thus inadvertent activation of the device). Preferably, the second dot 661 of the retainer 6 and the second dot 811 of the lock ring 8 abut against each when the lock ring 8 is in the locking position. Thus, the user has a feedback that the injection process is about to begin. This also helps prevent activation of the autoinjector 1 during a drop test.

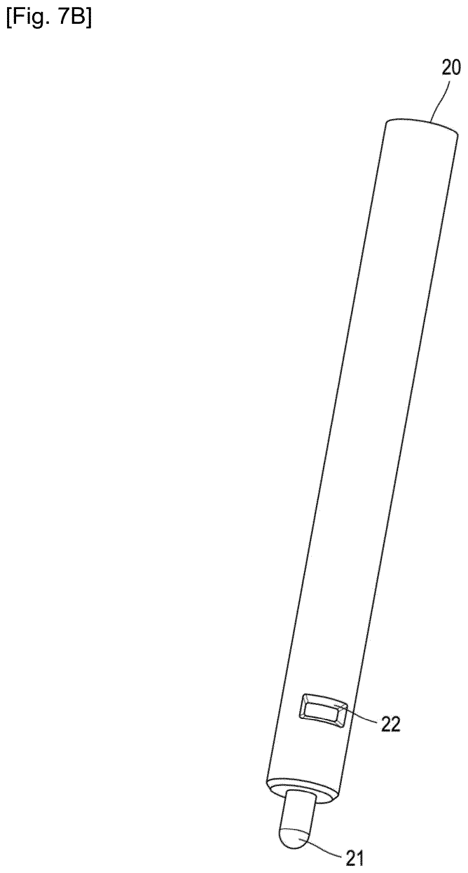

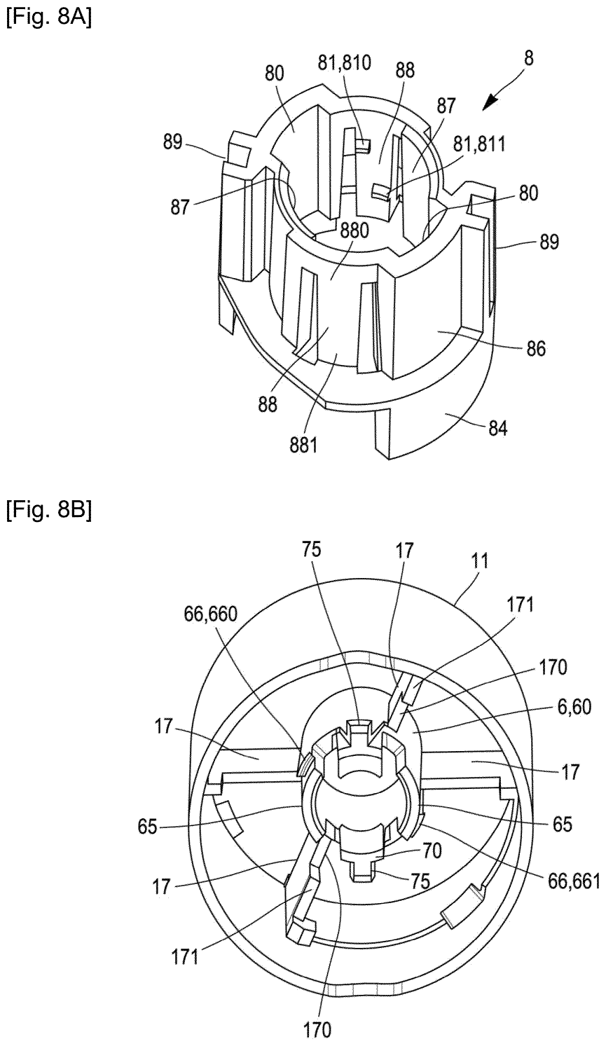

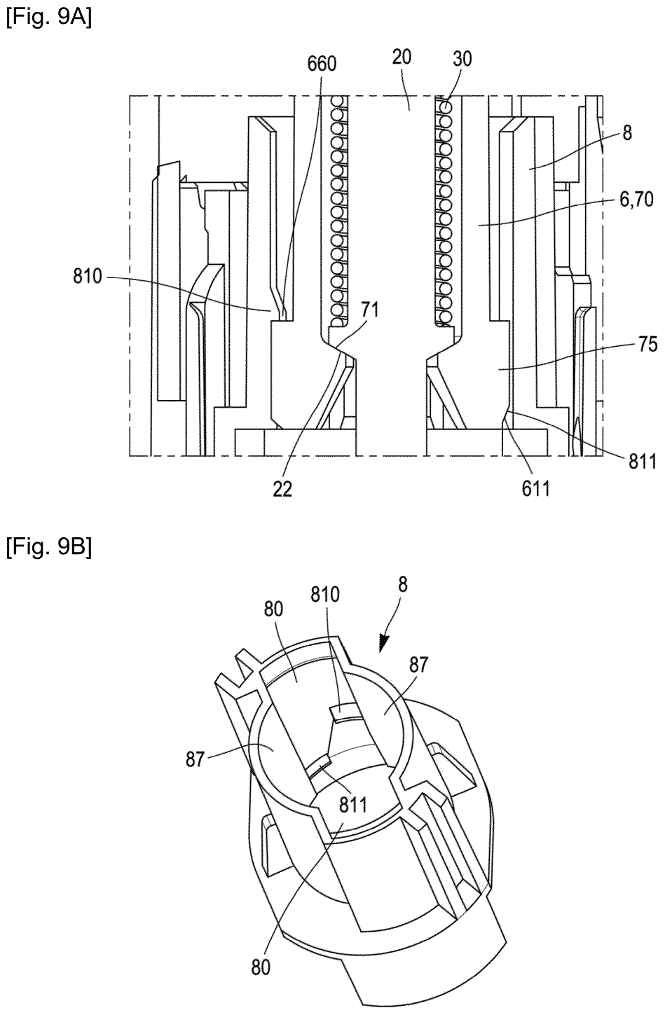

FIGS. 9A-9C illustrate maintaining means of an autoinjector 1 according to another embodiment. The maintaining means include a first blocking feature 660, 810 for blocking axial movement of the lock ring 8 in the distal direction, and a second blocking feature 661, 811 for blocking axial movement of the lock ring 8 in the proximal direction when a proximal force exerted on said lock ring 8 is below a predetermined threshold. The lock ring 8 has a first dot 810 and a second dot 811 which are arranged in each one of the axial grooves 80. That is, the first dot 810 and the second dot 811 are configured to engage the blocking legs 70 of the retainer 6, and more specifically the outward radial protrusion 75 of the blocking legs 70. The blocking legs 70 of the retainer 6 indeed have a first stop 660, which may be orthogonal to the longitudinal axis A, and a second stop 661, which may form a ramped surface at the distal end of the blocking legs 70 so that the second stop 661 however allows for axial movement of the lock ring 8 as soon as the pushing force exerted by the needle cover 40 on the lock ring 8 exceeds the predetermined threshold. In the locking position of the lock ring 8, the first stops 660 abut against the first dots 810 (thus preventing disassembly) while the second stops 661 abut against the second dots 811 (thus preventing inadvertent activation). The first stop 660 and the second stop 661 of the retainer 6 are here arranged on the blocking legs 70 so that the centering legs 65 of the retainer 6 may have a smooth outer surface devoid of any protrusion. The ramped surface formed by the second stop 661 enables to increase the resistance to drop tests, since any movement of the lock ring 8 in the proximal direction causes an inward deformation of the blocking legs 70 against the action of the injection spring 30 which is being compressed and which therefore acts against inadvertent activation of the device.

As visible in FIG. 9B, the lock ring 8 here has no flexible leg 88. Instead, the lock ring 8 has two enlarged centering surfaces 87 extending between the two axial grooves 80. The first dot 810 and the second dot 811 may be circumferentially shifted from each other; they are not axially aligned.

As illustrated in FIG. 9C, the outward radial protrusion 75 may include an axial slit 750 for improving the flexibility of the blocking legs 70. The outward radial protrusion 75 is thus divided in two parallel portions; the first stop 660 may be located on one of said two portions while the second stop 661 may be located on the other one of said two portions. It is also noted that the width w of the blocking legs 70 may here decrease towards their distal end. This may also apply to other embodiments of the autoinjector 1 for improving flexibility of the blocking legs 70.

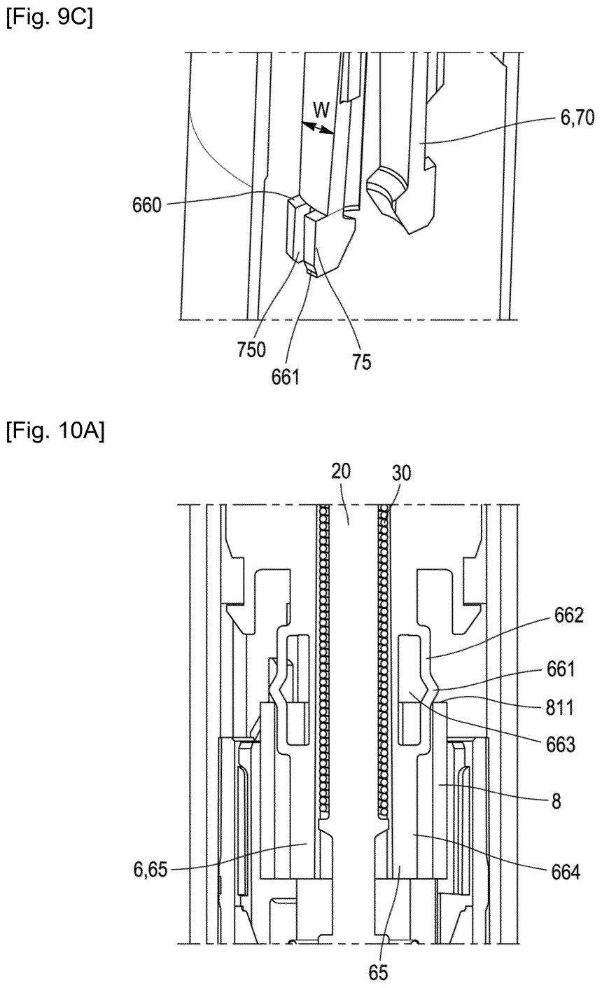

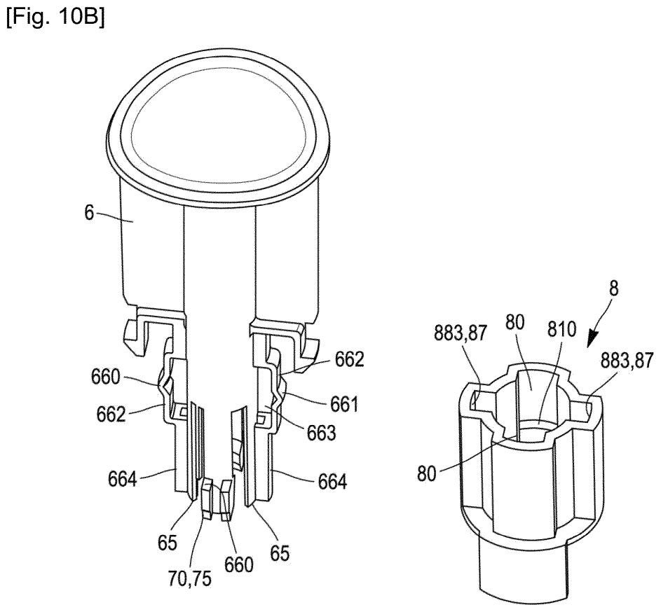

FIGS. 10A-10C illustrate maintaining means of an autoinjector 1 according to another embodiment. The maintaining means include a first blocking feature 660, 810 for blocking axial movement of the lock ring 8 in the distal direction, and a second blocking feature 661, 811 for blocking axial movement of the lock ring 8 in the proximal direction when a proximal force exerted on said lock ring 8 is below a predetermined threshold. The maintaining means of the lock ring 8 here include a proximal end of the lock ring 8 forming the second dot 811 for preventing proximal movement of the lock ring 8 (and thus inadvertent activation) and the retainer 6 having a second stop 661 arranged on a resilient arm 662. The proximal end (second dot 811) of the lock ring 8 may be configured to abut against the second stop 661 of the retainer 6 when the lock ring 8 is in the locking position, so as to provide feedback to a user that the autoinjector 1 is about to be activated and to limit risks of inadvertent activation during a drop test.

As illustrated in FIGS. 10A and 10B, the resilient arm 662 is arranged on the centering legs 65 and may be complementarily shaped with an axial groove 883 of the lock ring 8. The resilient arm 662 is configured to deform radially inwardly to allow the lock ring 8 to move towards the unlocking position under the pressure exerted by the needle cover 40. To that end, the arm 662 is separated from the sleeve by a radial gap 663. The arm 662 may have two opposite ends secured to the sleeve 60 so that the radial gap 663 forms a closed loop. As visible in FIGS. 10A and 10B, the centering legs 65 of the retainer 6 may further include, distal to the resilient arm 662, an axial rib 664 configured to complementarily engage the two diametrically opposite axial grooves 883 of the lock ring 8. The bottom of said axial grooves 883 thus forms the centering surfaces 87 of the lock ring 8. The axial grooves 80 of the lock ring 8 may have a radial protrusion forming the first dot 810 of the lock ring 8. In a similar manner as the embodiment illustrated in FIGS. 9A-9C, the first dot 810 of the lock ring 8 is configured to distally abut against the first stop 660 defined by the outward radial protrusion 75 of the blocking legs 70 (thus preventing disassembly).

The operation of the autoinjector 1 is described below.

Before activation of the autoinjector 1, the needle cover 40 is in the first extended position, the plunger rod 20 is in its initial position, the injection spring 30 is in its compressed state, the lock ring 8 is in the locking position and the blocking legs 70 are in their blocking (rest) position. The plunger rod 20 is thus prevented from moving in the distal direction by the inward radial protrusion 72 of the blocking legs 70 (FIGS. 5A, 5B).

The user first withdraws the cap 2 from the bottom body 12. This withdrawal entails removal of the neddle shield 102. During the cap removal, the fixed ring 4 may abut against a proximal shoulder 53 of the cam 50 to stop distal movement of the medical container 100.

The user then places the autoinjector 1 against the injection site. The distal end 42 of the needle cover 40 comes against the injection site. As the user presses the autoinjector 1 against the injection site, the needle cover 40 moves from the first extended position to the retracted position. This movement of the needle cover 40 in the proximal direction causes the tabs 41 of the needle cover 40 to slide against an upper ramp 511 of an oblique portion 510 of the first slot 51 of the cam 50. This causes the cam 50 to rotate so that the fixed ring 4 now axially faces a lower ramp 520 of the second slot 52 and so that the tabs 41 move at a proximal end 513 of the axial portion 512 of the first slot 51 of the cam 50. The proximal movement of the needle cover 40 towards the retracted position also causes the pushing surface 44 of the needle cover 40 to abut against the lock ring 8 and consequently to push the lock ring 8 in the proximal direction towards the unlocking position (FIG. 6A).

As a consequence, the radial outward protrusions of the blocking legs 70 go outside the axial grooves 80 of the lock ring 8. The blocking legs 70 are now free to deflect radially outwardly since the lateral blocking surface 82 of the lock ring 8 has been axially shifted away. Due to the distal force exerted by the injection spring 30 on the plunger rod 20, and due to the contact between the plunger rod 20 and the axial stop 71 of the blocking legs 70, the blocking legs 70 thus outwardly deflect. The axial stop 71 is moved away from the distal abutment surface 22 of the plunger rod 20, which can move towards the injection end position under the action of the injection spring 30. The plunger rod 20 coupled to the stopper 105 firstly pushes the medical container 100 so that the injection needle reaches the injection site and then pushes the stopper 105 inside the barrel 101 to expel the medical product. When the stopper 105 eventually abuts against the distal end 106 of the reservoir defined by the barrel 101, the injection is complete. An indicator, such as a visual, tactile or audible indicator, may inform the user that the injection is complete.

The user then moves the autoinjector 1 away from the injection site. The needle cover 40 slides in the axial portion 512 of the cam 50, moving from the retracted to the second extended (safety) position under the action of the safety spring 3. A locking mechanism may lock the needle cover 40 in the safety position to prevent needle stick injuries.

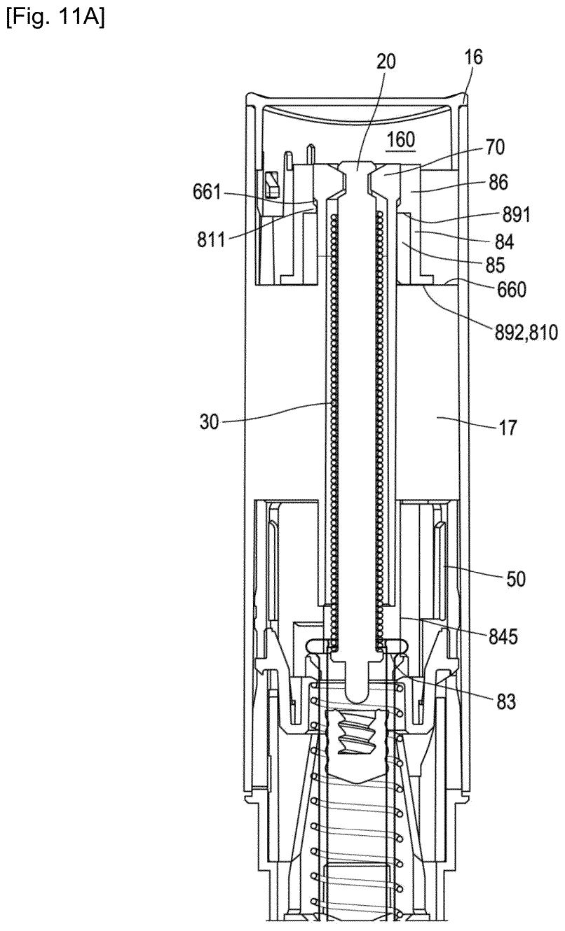

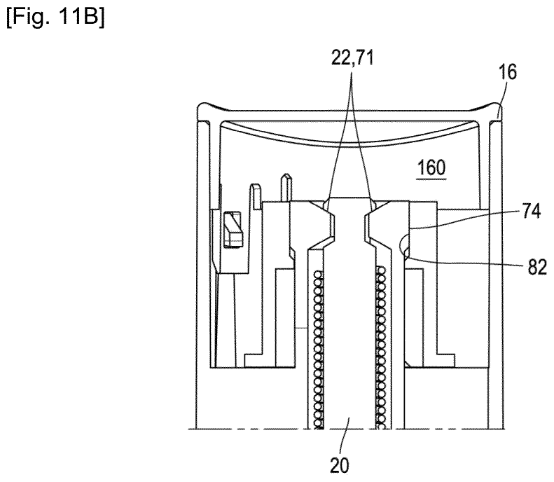

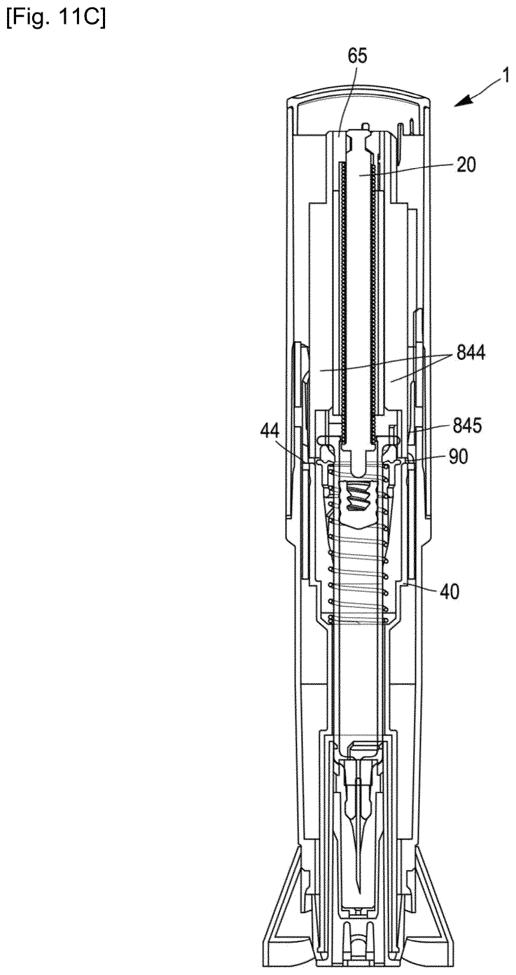

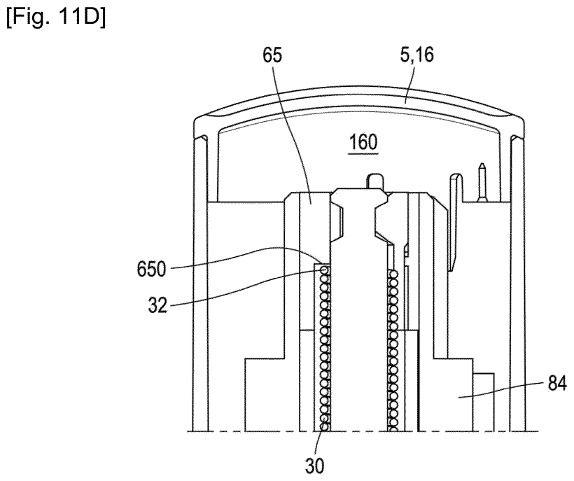

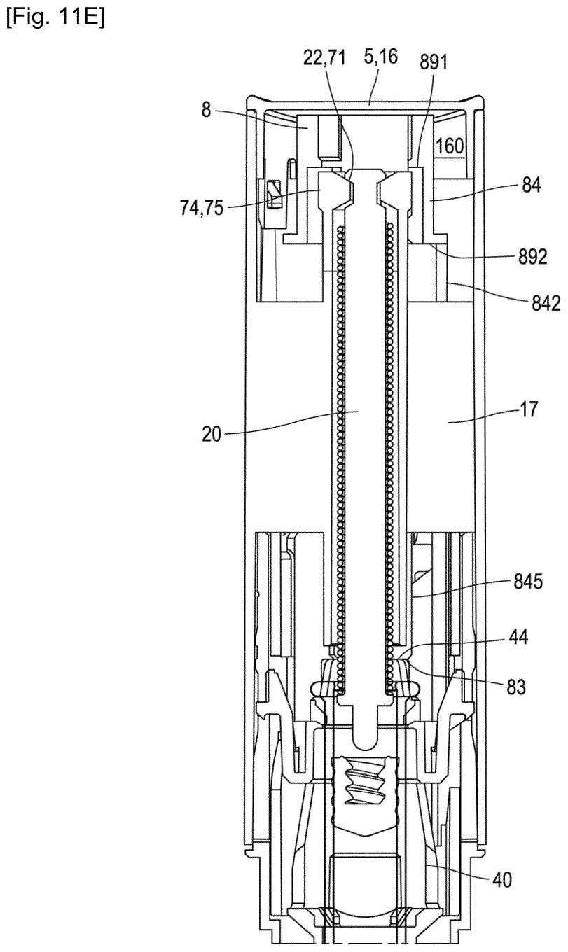

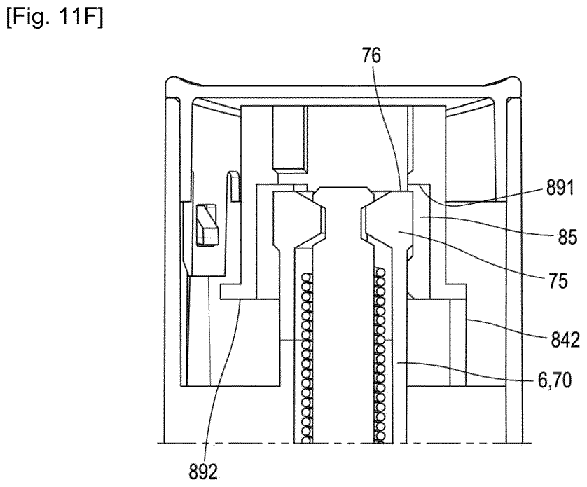

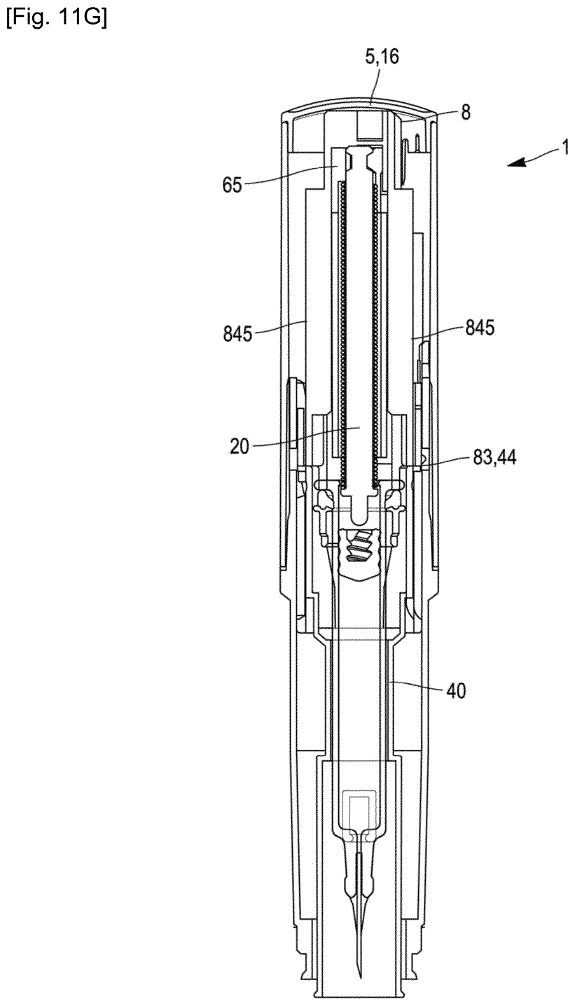

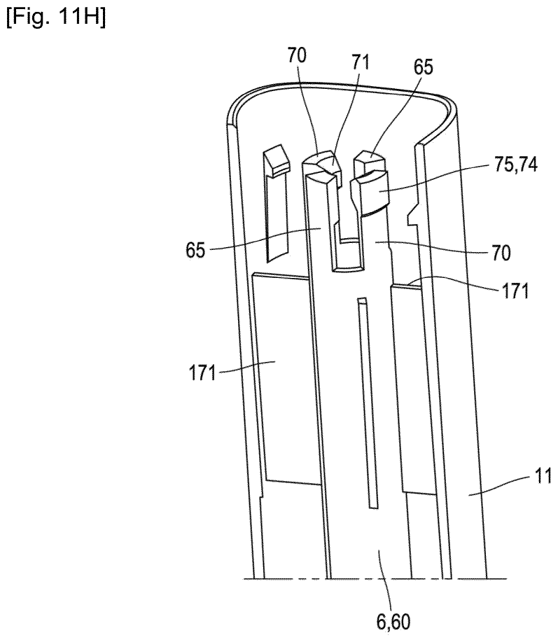

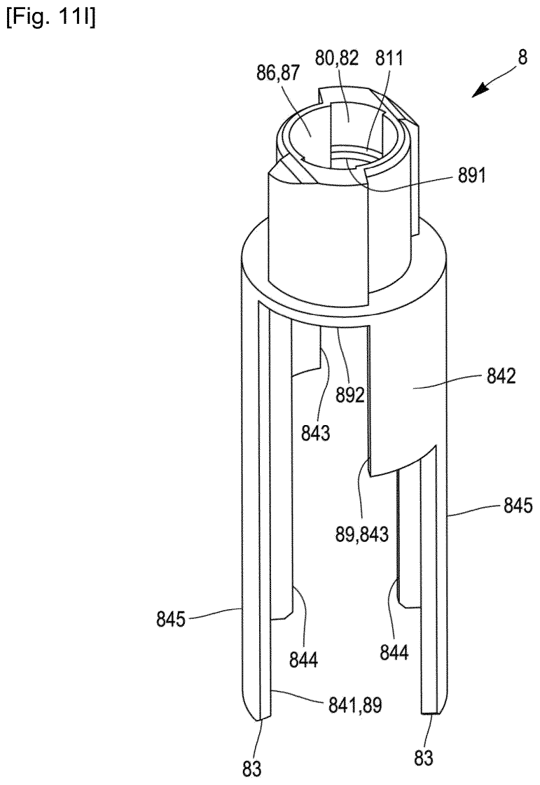

FIGS. 11A-11I illustrate an autoinjector 1 according to another embodiment. Similar features with the other embodiments are designated by the same numeral references. Instead of being located at the distal end of the retainer 6, the lock ring 8 here is arranged at the proximal end of the retainer 6. Besides, the retainer 6 here provides support for a proximal end of the injection spring 30. As visible in FIG. 11D, the centering legs 65 of the retainer 6 define a distal shoulder 650 serving as a support for the proximal end 32 of the injection spring 30, see FIGS. 11C, 11D. The autoinjector 1 also delimits a room 160 under its proximal end 16 for allowing the lock ring 8 to move to the unlocking position. In said unlocking position, the lock ring 8 may abut against the proximal end 16, more specifically the top cap 5 forming the proximal end 16 of the autoinjector 1.

The maintaining means include a first blocking feature 660, 810 for blocking axial movement of the lock ring 8 in the distal direction, and a second blocking feature 661, 811 for blocking axial movement of the lock ring 8 in the proximal direction when a proximal force exerted on said lock ring 8 is below a predetermined threshold. The maintaining means include a first dot 810 formed by a distal shoulder 892 of the lock ring 8 abutting against a first stop 660 formed by a proximal end of the connectors 17. Besides, the maintaining means include a second dot 811 formed by a radial protrusion in the axial grooves 80 of the lock ring 8 for abutting against a second stop 661 forming a ramped surface at a distal end of the blocking legs 70 and more specifically at the distal end of the outward radial protrusions 75.

With reference to FIGS. 11A and 11B, the lock ring 8 is in the locking position. The proximal end 63 of the tubular sleeve is opened and forms the blocking legs 70. The blocking legs 70 are movable between a blocking position which is a rest position (FIG. 11A), in which their axial stop 71 axially abuts against the distal abutment surface 22 of the plunger rod 20, and a release position which is a deformed position, in which the blocking legs 70 radially outwardly deflect so that their axial stop 71 is shifted away from the path of the plunger rod 20. Movement of the blocking legs 70 from the rest position to the deformed position is due to the injection spring 30 pushing the plunger rod 20 in the distal direction. The axial stop 71 of the blocking legs 70 may be arranged at a proximal side of an inward radial protrusion 72, which is here located at a proximal end 77 of the blocking legs 70. The distal end 73 of the blocking legs may be fixed to the sleeve 60. The blocking legs 70 may include a lateral blocking surface 74, which may be defined by an outward radial protrusion 75, for abutting against the lock ring 8, thereby maintaining the blocking legs 70 in their rest position.

With reference to FIGS. 11C and 11D, the tubular sleeve 60 may also include one or more axially extending centering legs 65 for providing support for the lock ring 8 so that the lock ring 8 can be centered with respect to the central longitudinal axis A and can slide along the sleeve 60. The centering legs 65 here do not include the connecting features 66 of the retainer 6, which are instead provided by the outward radial protrusion 75 and the connectors 17, as explained above. The centering legs 65 also include the distal shoulder 650 forming a support for the injection spring 30.

The connectors 17 may be in the form of radial walls extending parallel to the longitudinal axis A. As visible in FIG. 11H, the connectors 17 have a lateral surface forming a guideway 171 for guiding the proximal movement of the lock ring 8. More specifically, two edges 843 arranged on the lock ring 8 are configured to circumferentially abut against the guideway 171 for guiding the proximal movement of the lock ring 8.

As illustrated in FIG. 11C, the needle cover 40 in the first extended position and the lock ring 8 in the locking position may be axially separated by a predetermined axial gap 90. It should be noted that the needle cover 40 directly comes in abutment against the lock ring 8, as visible in FIG. 11G.

FIG. 11I illustrates the lock ring 8. The lock ring 8 is coupled to and axially movable with respect to the sleeve 60 between a locking position (FIGS. 11A-11D), and an unlocking position (FIGS. 11E, 11F), proximally located with respect to the locking position, in which the lock ring 8 allows the blocking legs 70 for moving to their release position. Movement of the lock ring 8 from the locking position to the unlocking position is caused by the pushing surface 44 of the needle cover 40 abutting against the distal abutment surface 83 of the lock ring 8. The distal abutment surface 83 of the lock ring 8 may be arranged at a distal end 840 of one or more, for instance two diametrically opposite, distally extending legs 845. The distal abutment surface 83 may be the distal-most surface of the lock ring 8.

Still with reference to FIG. 11I, the lock ring 8 includes a tubular wall defining an inner cavity 86 for receiving the retainer 6. An inner side of the tubular wall may define one or more centering surfaces 87 which are complementarily shaped with the centering legs 65 of the retainer 6. The tubular wall further defines one or more axial grooves 80 whose bottom delimits the lateral blocking surfaces 82 of the lock ring 8. The axial grooves 80 are thus shaped to receive the outward radial protrusion 75 of the blocking legs 70 and allow translation of the lock ring 8 along the blocking legs 70. As visible in FIG. 11A, the proximal portion (tubular wall 86) of the lock ring 8 is separated by the distal shoulder 891 and the distal portion of the lock ring 8 defines the radial room 85 for accommodating the outwardly deformed blocking legs 70. Here, the axial length of the distal portion (the protection walls 84) of the lock ring 8 may be longer than the axial length of the proximal portion (tubular wall 86) of the lock ring 8. As illustrated in FIG. 11I, the distal portion of the lock ring 8 further includes the axial legs 845 which distally protrude from the distal shoulder 892 so as to extend along the path of the needle cover 40 and transmit, by abutting against the needle cover 40, the axial movement of said needle cover 40 to the lock ring 8.

The lock ring 8 cannot rotate around the longitudinal axis A and moves in the axial direction only. The lock ring 8 further includes a guiding member 89 which here in the form of two circumferential walls 842 which define the two edges 843 for abutting and sliding against the guideway 171 formed by the lateral surface of the connectors 17. The edges 843 of the circumferential walls 842 block rotation of the lock ring 8 in a first direction. The opposite edge 841 of the legs 845 also circumferentially abuts against the connectors 17 for blocking rotation of the lock ring 8 in the opposite direction. The circumferential walls 842 may extend circumferentially from the legs 845, and they may be shorter than the legs 845 in the axial direction to avoid interference with other parts of the autoinjector 1. It is also noted that the lock ring 8 may include axial ribs 844 protruding from an inner surface of the protection walls 84 and configured to abut and slide against an outer surface of the sleeve 60. The axial ribs 844 provide axial guidance for the lock ring 8 while stiffening the protection walls 84.

As illustrated in FIG. 11F, in the unlocking position, the distal shoulder 891 of the lock ring 8 may be axially away from the outward radial protrusion 75 of the retainer 6, so that the inner distal shoulder 892 does not hinder movement of the blocking legs 70 towards their release (deformed) position. More specifically, in the unlocking position, the distal shoulder 891 is axially away from a proximal stop 76 of the blocking legs 70. This proximal stop 76 is configured to abut against the distal shoulder 891 of the lock ring 8 for preventing the lock ring 8 to move back to the blocking position when the needle cover 40 moves away from the lock ring 8 towards the safety position.

The autoinjector 1 of FIGS. 11A-11I may further include some or all of the other compatible features of the above-described embodiments.

It is readily understandable from the above description that the autoinjector 1 of the invention permits to reduce the steps that are necessary to trigger the injection (the user has no need to push a button and the lock ring 8 automatically slides when the autoinjector 1 is being pressed against the skin of the user). The effort required for activating the autoinjector 1 is lower than in the prior art because the needle cover 40 directly abuts against the lock ring 8 and they both move in the axial direction.

It is to be understood that the present invention is not limited to the embodiments described above and illustrated in the drawings; rather, the skilled person will recognize that many changes and modifications may be made within the scope of the appended claims.

1. An autoinjector, for automatic injection of a product into an injection site, said autoinjector comprising:
a housing extending along a longitudinal axis and configured to receive a medical container having a barrel defining a reservoir for containing a medical product, said barrel having a distal end provided with an injection needle and an opened proximal end configured to receive a plunger rod for pushing a stopper arranged inside the barrel,
a needle cover coupled to and axially movable with respect to said housing between a first extended position, in which the needle cover at least partially shields the injection needle, a retracted position, in which the needle cover moves proximally with respect to the housing, and a second extended position in which the needle cover moves back in the distal direction to shield the injection needle,
a plunger rod axially movable inside the housing between an initial position and an injection end position distally located relative to said initial position, the plunger rod being configured to push the stopper in order to expel the medical product when moving from the initial position to the injection end position,
biasing member for biasing the plunger rod in a distal direction towards the injection end position,
a retainer, fixed relative to the housing, for maintaining the plunger rod in the initial position against the action of the biasing member, the retainer including a resilient blocking member radially deformable between a blocking position, in which the resilient blocking member axially abuts against a distal abutment surface of the plunger rod for blocking the plunger rod in the initial position, and a release position, in which the resilient blocking member is away from the distal abutment surface of the plunger rod to allow for movement of the plunger rod in the distal direction,
a lock ring arranged on the fixed retainer and axially movable with respect to the resilient blocking member of the retainer so that the lock ring can slide without rotating along the retainer, between a locking position, in which the lock ring radially abuts against a lateral blocking surface of the resilient blocking member to maintain the resilient blocking member in the blocking position, and an unlocking position, in which the lock ring is axially shifted away from the lateral blocking surface of the resilient blocking member, axial movement of the lock ring from the locking to the unlocking position being caused by the needle cover moving from the first extended position to the retracted position.

2. The autoinjector according to claim 1, wherein the needle cover has a pushing surface configured to abut against a distal abutment surface of the lock ring to move said lock ring in the unlocking position when the needle cover moves towards the retracted position.

3. The autoinjector according to claim 2, wherein the pushing surface the needle cover and the distal abutment surface of the lock ring are axially at a distance from each other before use of the autoinjector.

4. The autoinjector according to claim 1, wherein the blocking position of the resilient blocking member is a rest position.

5. The autoinjector according to claim 1, wherein the retainer is an axial sleeve arranged within the housing and the resilient blocking member is an axially extending resilient blocking leg forming a distal end or a proximal end of said axial sleeve, said blocking leg including an inward radial protrusion, which defines an axial stop for abutting against the distal abutment surface of the plunger rod, and an opposite outward radial protrusion, which defines the lateral blocking surface for abutting against the lock ring.

6. The autoinjector according to claim 1, wherein the lock ring includes a proximal portion and a distal portion separated by a distal shoulder, and the distal portion has a larger inner diameter D1 than the inner diameter D2 of the proximal portion.

7. The autoinjector according to claim 5, wherein the distal shoulder of the lock ring and the outward radial protrusion of the resilient blocking member the retainer are at an axial distance from each other when the lock ring is in the unlocking position and the resilient blocking member is in the release position.

8. The autoinjector according to claim 5, wherein the outward radial protrusion of the resilient blocking member includes a proximal stop configured to abut against the distal shoulder of the lock ring for preventing the lock ring to move back to the blocking position.

9. The autoinjector according to claim 1, wherein the lock ring includes one or more axial protection walls defining a radial room for receiving the resilient blocking member the release position.

10. The autoinjector according to claim 1, wherein the lock ring defines an inner cavity for receiving the retainer, said inner cavity defining a centering surface for sliding against the retainer and guiding the movement of the lock ring along the longitudinal axis, and an axial groove defining a lateral blocking surface for blocking the resilient blocking member of the retainer in the blocking position.

11. The autoinjector according to claim 1, wherein the lock ring is maintained assembled to the retainer.

12. The autoinjector according to claim 11, further comprising at least a first blocking feature for blocking axial movement of the lock ring in the distal direction, and a second blocking feature for blocking axial movement of the lock ring in the proximal direction when a proximal force exerted on said lock ring is below a predetermined threshold.

13. The autoinjector according to claim 2, wherein the distal abutment surface of the lock ring is arranged at a distal end of said lock ring and the pushing surface of the needle cover is arranged at a proximal end of said needle cover.

14. The autoinjector according to claim 2, wherein the needle cover has a radial tab engaged in a first slot of a rotatable cam, and the radial tab has a proximal surface which at least partially forms the pushing surface of the needle cover.

15. The autoinjector according to claim 1, wherein the lock ring has a guiding member for preventing rotation of said lock ring around the longitudinal axis.
